(19) United States
(12) Patent Application Publication
(10) Pub. No.: US 2008/0193527 A1
(43) Pub. Date: Aug. 14, 2008

(54) PHARMACEUTICAL COMPOSITIONS CONTAINING QUETIAPINE FUMARATE

(75) Inventors: Anna Ruiz Amenos, Barcelona (ES); Carmen Ubeda Perez, Cabrils. Barcelona (ES); Ignacio Diez Martin, Barcelona (ES)

Correspondence Address: INTELLECTUAL PROPERTY / TECHNOLOGY LAW, PO BOX 14329, RESEARCH TRIANGLE PARK, NC, 27709, US

(73) Assignee: LABORATORIOS LESVI, SL, Barcelona (ES)

(21) Appl. No.: 11/737,462

(22) Filed: Apr. 19, 2007

(30) Foreign Application Priority Data
Feb. 14, 2007 (EP) 07380037.7

(51) Int. Cl. A61K 9/22; A61K 31/553

(52) U.S. Cl. 424/465; 424/464; 424/468; 514/211.13

(57) ABSTRACT

A granule formulation useful for preparation of pharmaceutical compositions. The granule formulation includes a core containing quetiapine or a pharmaceutically acceptable salt thereof as an active ingredient, and a binder agent. The core is coated with a coating layer including a lubricant agent. Solid pharmaceutical compositions containing quetiapine, and their preparation, are described.

### CROSS-REFERENCE TO RELATED APPLICATION

The priority of European Patent Application EP07380037.7 filed Feb. 14, 2007 is hereby claimed under the provisions of 35 USC § 119.

### FIELD OF THE INVENTION

The present invention relates to new pharmaceutical compositions for the oral administration of Quetiapine or a pharmaceutically acceptable salt thereof, and to a process for its manufacture.

### BACKGROUND OF THE INVENTION

Quetiapine is a compound of formula (I):

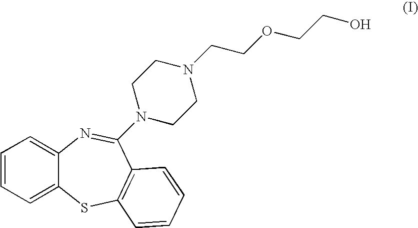

which has been employed as an antipsychotic or neuroleptic agent in the treatment of schizophrenia and bipolar mania, due to its antidopaminergic activity.

Quetiapine is currently marketed as a hemifumarate salt in the form of tablets of several doses of 25 mg, 100 mg, 200 mg and 300 mg for the administration two or three times per day. However, previously described formulations of quetiapine have certain drawbacks derived from the poor dissolution properties of this medicament and the uncontrolled release profile provided by said formulations. For example, document WO2005/041935 describes Quetiapine formulations which do not provide a constant or substantially constant level of quetiapine, such that the patient can, at certain time intervals, receive therapeutic amounts of quetiapine exceeding the recommended doses, whereas at other times the amount may be below the therapeutically effective limits.

Patent applications WO97/45124 and WO2005/041935 describe modified-release pharmaceutical compositions containing Quetiapine, i.e. they slowly release the active ingredient in long time intervals. For example patent WO2005/041935 describes solid dosage pharmaceutical compositions comprising a matrix formed by means of melted waxes, whereas application WO97/45124 describes the use of matrices with a gelling agent. However, in the later application, the use of water-soluble active ingredients, such as quetiapine or its pharmaceutically acceptable salts, combined with gelling agents such as hydroxypropylmethylcelluloses, can give rise to a phenomenon known as dumping in which the release of the active ingredient is delayed but once it starts the release occurs at very high rates.

Patent EP1218009 describes the preparation of granules containing Quetiapine and a freely or very water-soluble binder for their use in suspensions or solutions. Patent WO03/039516 relates to methods for improving the dissolution of poorly dispersible medicaments among them Quetiapine is included. The dissolution is improved by means of preparing granules in which a floating agent is added to the medicament. However, there is no indication about the release profile of these medicaments in the granulate formulations.

For all these reasons, there is still a need for developing pharmaceutical compositions which incorporate Quetiapine or one of its pharmaceutically acceptable salts or methods for preparing said compositions with an improved physical stability which allows their marketing without the active ingredient release properties being affected.

### BRIEF DESCRIPTION OF THE INVENTION

The aim of the present invention is to provide pharmaceutical compositions containing quetiapine or a pharmaceutically acceptable salt thereof for oral administration having an improved dissolution profile and an improved physical stability without affecting the release profile of said active ingredient. In addition, it is an aim of the present invention to provide a process for preparing said pharmaceutical compositions, particularly tablets, by means of a process that can be applied at an industrial level with low energy costs and which does not subject the active ingredient to aggressive formulation conditions which can entail the loss of stability of the product.

The authors of the present invention have found that the use of granules containing quetiapine or a pharmaceutically acceptable salt thereof and coated with a lubricating agent allows preparing oral pharmaceutical compositions, particularly in the form of tablets, with an improved physical stability without affecting the dissolution properties of the oral compositions, making them suitable for therapeutic use.

Accordingly, a first aspect of the present invention is a granule formulation for the preparation of pharmaceutical compositions comprising:
a) a core comprising quetiapine or a pharmaceutically acceptable salt thereof as active ingredient, and a binder agent; and
b) a coating layer comprising a lubricant agent.

In a particular embodiment of the invention, the core may further comprise a diluent agent and/or a disintegrant agent.

A second aspect of the present invention is a process for preparing a granule formulation as defined above, comprising:
a) providing quetiapine or a pharmaceutically acceptable salt thereof, optionally in mixture with a disintegrant agent and/or a diluent agent, as an active ingredient composition;
b) combining the active ingredient composition with binder agent and solvent to form a mixture, comprising a combination sequence selected from among (i) and (ii):
   (i) adding the binder agent to the active ingredient composition to form a binder-containing composition, and adding solvent to the binder-containing composition to form said mixture; and
   (ii) adding a solution or suspension comprising the binder agent and solvent to the active agent composition to form said mixture;
c) wet granulating said mixture to form granules;
d) drying said granules to form dried granules;
e) sieving said dried granules to recover product granules; and
f) coating the product granules with a lubricant agent, to yield said granule formulation.

Another aspect of the present invention relates to the use of the granule formulation as defined above for the elaboration of pharmaceutical compositions.

Another aspect of the invention refers to a pharmaceutical composition comprising the granule formulation as defined above, optionally in combination with one or more pharmaceutically acceptable excipients. In a particular embodiment, the pharmaceutical composition is in the form of a tablet.

Further, in another aspect the invention refers to an immediate release tablet which comprises the granule formulation as defined above wherein the quantity of the lubricant agent is between 5 and 10% by weight with respect to the total weight of the granule formulation.

In still another aspect the invention relates to a sustained release tablet which comprises the granule formulation as defined above wherein the quantity of the lubricant agent is between 15 and 25% by weight with respect to the total weight of the granule formulation.

Finally, another aspect of the invention is a process for the preparation of an immediate or sustained release tablet as defined above comprising:
a) preparing a granule formulation in accordance with the invention;
b) optionally mixing the granule formulation obtained in step a) with one or more pharmaceutically acceptable excipients;
c) tableting the granule formulation obtained in step a) or in its case the mixture obtained in step b); and
d) coating the tablets obtained in step c).

### DETAILED DESCRIPTION OF THE INVENTION

In the present invention as “granule formulation” it is understood to refer to a set of granules, each of them comprising a core which comprises quetiapine as the active ingredient and a binder agent, said core being coated by a layer comprising a lubricating agent. In addition, the core of the granules may optionally comprise at least one diluent agent and/or a disintegrant agent.

Unless otherwise indicated, “quetiapine” is understood to be the compound quetiapine or a pharmaceutically acceptable salt thereof, preferably quetiapine fumarate with a 2:1 stoichiometry, also known as quetiapine hemifumarate. Quetiapine may be incorporated in the granules in crystalline form either as a free compound or as a solvate. For example, in the case of using quetiapine fumarate, it can be incorporated in any of the several polymorphic forms described in patent applications WO99/06381, WO03/080065 and WO2004/078735.

Quetiapine is preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

The binder agent included in the core of the granules is selected from povidone, cornstarch, hydroxypropylcellulose and copovidone. Advantageously, the use of povidone K-25 as a binder is preferred. The amount of binder agent to be added to the core may vary between 1 and 15% by weight with respect to the total weight of the granule.

The diluent agent optionally included in the core is preferably selected from microcrystalline cellulose, lactose monohydrate and dibasic calcium phosphate. Advantageously, the use of microcrystalline cellulose as a diluent agent is particularly preferred. The amount of the diluent agent to be optionally added to the core may vary between 10 and 40% by weight with respect to the total weight of the granule.

The disintegrant agent optionally included in the core of the granule is preferably selected from sodium starch glycolate, crospovidone and sodium croscarmellose. Advantageously, sodium starch glycolate type A, known with the commercial name Primogel®, as a disintegrant agent is particularly preferred. The amount of disintegrant agent to be optionally added to the core may vary between 3 and 20% by weight with respect to the total weight of the granule.

For coating the core of the granule, a lubricating agent selected from the glyceryl behenate, glyceryl palmitostearate and macrogol group can be used. Advantageously, glyceryl behenate as a lubricating agent is particularly preferred. The amount of lubricant agent to be used for preparing the coating layer may vary between 5 and 25% by weight with respect to the total weight of the granule.

In a particular embodiment of the invention, the granule formulation is based on a set of granules, said granules comprise a core containing quetiapine hemifumarate combined with microcrystalline cellulose as a diluent agent, sodium starch glycolate (Primogel®) as a disintegrant agent and Povidone (PVP K25) as a binder, said core being coated with a glyceryl behenate layer as a lubricating agent.

The granule formulation described above can be prepared by any method known in the state of the art. However, in a particular embodiment, said granule formulation is prepared by wet granulation of quetiapine with a binder and optionally with a diluent agent and/or a disintegrant agent, followed by a coating process by means of a lubricant agent. Accordingly, the process for preparing a granule formulation as described above comprises the steps of:
a) providing quetiapine or a pharmaceutically acceptable salt thereof and, optionally mixing it with a disintegrant agent and/or a diluent agent;
b) adding to the quetiapine or to the mixture obtained in step a) a binder agent;
c) adding a solvent to the mixture obtained in step b), or optionally, adding a solution or suspension containing a binder and a solvent to the quetiapine or to the mixture obtained in step a), thus suppressing step b);
d) wet granulating the mixture obtained in step c);
e) drying the granules obtained in step d);
f) sieving the dried granules obtained in step e); and
g) coating the granules with a lubricant agent.

The first step consists of providing quetiapine or a pharmaceutically acceptable salt thereof and optionally mixing the active ingredient quetiapine with a disintegrant and/or a diluent.

Quetiapine or a pharmaceutically acceptable salt thereof, such as fumarate, can be prepared according to the method described in patent application WO2005/014590, which is incorporated herein as a reference. The amount of quetiapine in the granules is comprised between 20 and 80% of the total weight of the granule formulation, preferably between 40 and 80%.

The amount of disintegrant to be optionally added is comprised between 3% and 20% by weight with respect to the total weight of the granule formulation. Preferably, between 5% and 15% is added, more preferably between 7% and 12% of the total weight of the granule formulation. The amount of diluent to be optionally added is comprised between 10% and 40% by weight with respect to the total weight of the granule formulation, preferably between 15% and 25% of the total weight.

In the second step, the amount of binder to be added to the quetiapine or to the mixture obtained in step a) is comprised between 1% and 15% by weight with respect to the total weight of the granule formulation.

In the third step, a solvent is added in an amount comprised between 25% and 65% by weight with respect to the weight of mixture to be granulated, which will serve to carry out the wet mixture. Alternatively, the binder agent can be previously dissolved or suspended in said solvent and then added to the quetiapine or to the mixture obtained in step a) thus suppressing step b). As solvents for preparing the wet mixture, water, hydroalcoholic mixtures and alcohols can be used, being preferred the use of water as a solvent for the granulation of the mixture. This solvent is later eliminated from the composition by means of a drying step.

Then, in the fourth step, the wet granulation is performed. The granules can be produced by a known granulation method such as rolling granulation, fluidized-bed granulation, stirring granulation and the like. Additionally, suitable equipment for this type of processing can be used, for example, the granulation can be carried out in a low shear mixer or a high shear mixer. However, a low shear granulation will be preferably used since pharmaceutical compositions with a faster dissolution profile are obtained.

Once the wet granules are obtained, they are subjected to a drying process to eliminate the solvent. This step can be carried out for example in a fluid bed dryer. The granules are subjected to a temperature comprised between 40° C. and 90° C., preferable between 60° C. and 80° C., for the time period necessary to obtain granules with a moisture content less than 5%, preferably less than 3%.

Subsequently, the dried granules are calibrated by sieving or milling.

Finally, the sieved or milled granules are coated with a lubricating agent. The coating process can be carried out by mixing the granules with the lubricating agent by any process known by a skilled person.

Surprisingly, the inventors have discovered that the amount of lubricating agent used for coating the granules allows controlling the rate of release of the active ingredient. Thus, the use of a lubricating agent as described above for coating the core of the granules in a proportion between 5% and 10% by weight with respect to the total weight of the granule allows preparing immediate release compositions. On the contrary, the use of the coating lubricating in a proportion between 15% and 25% by weight with respect to the total weight of the granule allows preparing sustained release compositions.

By “immediate release” it is understood a release form in which greater than or equal to about 50% or more, preferably about 75% of quetiapine is released within two hours of administration, preferably within one hour of administration.

By “sustained release” it is understood a release form in which quetiapine is released at such a rate that blood (e.g. plasma) levels are maintained within a therapeutic range but below toxic levels for at least 8 hours, preferably at least about 12 hours after administration.

Accordingly, the granule formulation of the invention can be used for the elaboration of pharmaceutical compositions with different release profiles. Examples of these pharmaceutical compositions include any solid (tablets, pills, capsules, etc.) or liquid (solutions, suspensions or emulsions) composition for oral administration. In an embodiment of the invention, the pharmaceutical composition is an immediate release composition. In another embodiment, the pharmaceutical composition is a sustained release composition.

Suitable dose forms for oral administration may further contain conventional excipients known in the art such as binding agents, for example syrup, acacia, cellulose derivatives (i.e. hydroxypropylcellulose, carboxymethylcellulose, etc.) gelatin, sorbitol, tragacanth, or polyvinylpyrrolidone; fillers, for example lactose, sugar, maize starch, calcium phosphate, sorbitol or mannitol; tabletting lubricants, for example magnesium stearate; disintegrants, for example starch, crospovidone, sodium starch glycolate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The mentioned formulations will be prepared using standard methods such as those described or referred to in the Spanish and US Pharmacopoeias and similar reference texts.

In a preferred embodiment of the invention, the pharmaceutical composition is in the form of a tablet. The excipients used for preparing tablets may comprise between 0.25% and 5% by weight with respect to the total weight of the tablet of one or more lubricating agents, between 5% and 20% by weight of one or more disintegrants, between 20% and 50% by weight of one or more diluents and between 0.1% and 0.5% by weight of a glidant.

As disintegrant, low-substituted hydroxypropylcellulose, hydroxyethylcellulose, crospovidone, croscarmellose, starch, sodium carboxymethyl starch, casein derivatives or mixture thereof can be used.

As lubricating agent, magnesium stearate, calcium stearate, glyceryl palmitostearate, talcum, stearic acid, glyceryl behenate, sodium lauryl sulfate, sodium stearyl fumarate or mixtures thereof can be used. A stearate will preferably be used, still more preferably, magnesium stearate.

As diluent, a saccharide (monosaccharide or oligosaccharide, polysaccharides) and/or their oxidized and/or reduced forms; lactose in its anhydrous, monohydrate, agglomerated or spray forms; mannitol; cellulose powder, microcrystalline cellulose, silicified microcrystalline cellulose or chemically modified cellulose derivatives, such as hydroxypropylcellulose, hydroxypropylmethylcellulose; starch, sucrose, pharmaceutically acceptable inorganic compounds such as dibasic calcium phosphate, calcium or magnesium carbonates, magnesium oxide, or mixtures thereof can be used.

Additionally, previously prepared coprocessed diluents can be used such as Cellactose®, coprocessed lactose and cellulose powder, or Microcellac®, coprocessed lactose and microcrystalline cellulose, among others.

Preferably, cellulose will be used, more preferably, microcrystalline cellulose.

As glidant, anhydrous or hydrated colloidal silica, magnesium trisilicate or talc can be used.

The authors of the present invention have been able to prove that when quetiapine or one of its pharmaceutically acceptable salts are mixed with the necessary excipients and are compressed directly with no other type of processing (e.g., granulation, etc.), the use of the direct compression method gives rise to very adherent tablets. It has also been proved that when quetiapine granules which are not coated with a lubricating agent are prepared, the obtained tablets are still adherent.

However, upon coating the granules with a lubricating agent, adhesions are minimized or disappear, providing tablets with a regular surface.

An additional aspect of the invention refers to an immediate release tablet which comprises a granule formulation as described previously wherein the quantity of lubricant agent is between 5% and 10% by weight with respect to the total weight of the granule formulation. Further another aspect of the invention relates to a sustained release tablet which comprises a granule formulation as described previously wherein the quantity of lubricant agent is between 15% and 25% by weight with respect to the total weight of the granule formulation.

Finally, another aspect of the present invention consists of providing a process for preparing an immediate or sustained release tablet as defined above which comprises:
1. preparing a granule formulation according to the process previously 1 0 described;
2. optionally mixing the granule formulation obtained in step 1) with one or more pharmaceutically acceptable excipients;
3. tableting the mixture obtained in step b); and
4. coating the tablets obtained in step c).

Step 1) comprises all the steps a) to g) previously described in the preparation of the granule formulation of the invention. In the case of preparing an immediate release tablet, the amount of lubricating agent to be used for coating the core of the granules may vary between 5% and 10% by weight with respect to the total weight of the granule. However, in the case of preparing a sustained release tablet, the amount of lubricating agent to be used for coating the core of the granules may vary between 15% and 25% by weight with respect to the total weight of the granule

The excipients optionally used in step 2) for preparing tablets may comprise between 0.25% and 5% by weight with respect to the total weight of the tablet of one or more lubricating agents, between 5% and 20% by weight of one or more disintegrants, between 20% and 50% by weight of one or more diluents and between 0.1% y un 0.5% by weight of a glidant.

The tableting process of step 3) can be carried out by any method known in the state of the art for preparing tablets, including for example direct compression, double compression, granulation etc. Finally, the tablet is coated with a conventional or enteric coating material or a polymeric coating material. For example, a mixture of hypromellose, titanium dioxide and macrogol in purified water can be used.

The pharmaceutical compositions of this invention may be used with other drugs to provide a combination therapy. The other drugs may form part of the same composition, or be provided as a separate composition for administration at the same time or at different time.

In the following, the present invention is further illustrated by examples. They should in no case be interpreted as a limitation of the scope of the invention as defined in the claims.

### EXAMPLES

### Example 1

### Preparation of Quetiapine Tablets

Quantitative Composition:

| | % |
|---|---|
| Quetiapine hemifumarate | 37 |
| Lactose monohydrate | 18 |
| Microcrystalline Cellulose | 18 |
| (Avicel PH102) | |
| Povidone (K-25) | 3 |
| Na starch glycolate type A (Primojel) | 15 |
| Glyceryl behenate | 5 |
| Anhydrous colloidal silica (Aerosil) | 0.3 |
| Magnesium stearate | 1 |
| Purified watert* | 26* |
| Coating dispersion | 3** |

*solvent which disappears during the manufacturing process.
**dry residue

### Detailed Description of the Manufacturing Process:

Quetiapine hemifumarate is mixed with povidone and 50% of the total sodium starch glycolate. The mixture is granulated in a low shear mixer with purified water, dried and sieved. The obtained granules are coated with glyceryl behenate by mixing. The coated granules are mixed with the remaining 50% of sodium starch glycolate, together with microcrystalline cellulose, lactose and aerosil and finally with magnesium stearate. The obtained mixture is compressed and the tablets are coated with a coating dispersion formed by traditional coating agents.

### Example 2

Quantitative Composition:

| | % by weight |
|---|---|
| Quetiapine hemifumarate | 37 |
| Lactose monohydrate | 20 |
| Microcrystalline cellulose (Avicel PH102) | 20 |
| Povidone (K-25) | 3 |
| Na starch glycolate type A (Primojel) | 11 |
| Glyceryl behenate | 5 |
| Anhydrous colloidal silica (Aerosil) | 0.3 |
| Magnesium stearate | 1 |
| Purified water* | 31* |
| Coating dispersion | 3** |

*solvent which disappears during the manufacturing process.
**dry residue

### Detailed Description of the Manufacturing Process:

Quetiapine hemifumarate is mixed with povidone, 50% of the total sodium starch glycolate and 50% of the total microcrystalline cellulose. The mixture is granulated in a low shear mixer with purified water, dried and sieved. The obtained granules are coated with glyceryl behenate by mixing. The coated granules are mixed with the remaining 50% of microcrystalline cellulose and sodium starch glycolate, together with lactose and aerosil and finally with magnesium stearate. The obtained mixture is compressed and the tablets are coated with a coating dispersion formed by traditional coating agents.

### Example 3

Quantitative Composition:

| | % by weight |
|---|---|
| Quetiapine hemifumarate | 37 |
| Lactose monohydrate | 22 |
| Microcrystalline cellulose (Avicel PH102) | 22 |
| Povidone (K-25) | 3 |
| Na starch glycolate type A (Primojel) | 7 |
| Glyceryl behenate | 5 |
| Anhydrous colloidal silica (Aerosil) | 0.3 |
| Magnesium stearate | 1 |
| Purified water* | 29* |
| Coating dispersion | 3** |

*solvent which disappears during the manufacturing process.
**dry residue

### Detailed Description of the Manufacturing Process:

Quetiapine hemifumarate is mixed with povidone, 50% of the total sodium starch glycolate and 50% of the total microcrystalline cellulose. The mixture is granulated in a low shear mixer with purified water, dried and sieved. The obtained granules are coated with glyceryl behenate by mixing. The coated granules are mixed with the remaining 50% of microcrystalline cellulose and sodium starch glycolate, together with lactose and aerosil and finally with magnesium stearate. The obtained mixture is compressed and the tablets are coated with a coating dispersion formed by traditional coating agents.

### Example 4

Quantitative Composition:

| | % by weight |
|---|---|
| Quetiapine hemifumarate | 58 |
| Lactose monohydrate | 16 |
| Microcrystalline cellulose (Avicel PH102) | 16 |
| Povidone (K-25) | 5 |
| Na starch glycolate type A (Primojel) | 5 |
| Magnesium stearate | 1 |
| Purified water* | 38* (Meth. A) |
| | 43* (Meth. B) |

*solvent which disappears during the manufacturing process.

The formula described in example 4 was manufactured by two different methods, one wherein the granulation is carried out using a high shear mixer (Method A) and other wherein a low shear mixer is used (Method B).

The dissolution profiles of the obtained tablets were determined in hydrochloric acid (900 ml, Ph. Eur. paddle apparatus, 50 rpm). The obtained results are indicated in the following table:

% DISSOLVED

| TIME | Method A | Method B |
|---|---|---|
| 15 | 37 | 97 |
| 30 | 72 | 101 |
| 45 | 90 | 101 |
| 60 | 99 | 103 |

By using a low shear mixer during the granulation process, a faster dissolution profile is obtained, allowing a total dissolution of the tablet at least 30 minutes before than the tablet containing granules manufactured with a high shear mixer.

### Example 5

Quantitative Composition:

| | Formula A (%) | Formula B (%) |
|---|---|---|
| Quetiapine hemifumarate | 38 | 38 |
| Lactose monohydrate | 20 | 18 |
| Microcrystalline cellulose | 21 | 19 |
| (Avicel PH102) | | |
| Dibasic calcium phosphate | 1 | 1 |
| Povidone (K-25) | 3 | 3 |
| Na starch glycolate type A (Primojel) | 15 | 15 |
| Glyceryl behenate | — | 5 |
| Magnesium stearate | 1 | 1 |
| Purified water* | 28* | 28* |

*solvent which disappears during the manufacturing process.

Formulas A and B were manufactured following the method described in Example 1. The difference between both formulas is in the coating of the obtained granules; in formula B the granules are coated with glyceryl behenate by mixing, while in formula A the granules are not coated. Adherent compounds are obtained in the compression of formula A; the coating of the granules with glyceryl behenate solves the adhesion problems in the tablets.

1. A granule formulation for the preparation of pharmaceutical compositions, each granule comprising:
a) a core comprising (i) quetiapine, or a pharmaceutically acceptable salt thereof, as active ingredient, and (ii) a binder agent; and
b) a coating layer comprising a lubricant agent.

2. The granule formulation according to claim 1 wherein the core further comprises a diluent agent and/or a disintegrant agent.

3. The granule formulation according to claim 1 wherein the binder agent is selected from the group consisting of povidone, corn starch, hydroxypropylcellulose and copovidone.

4. The granule formulation according to claim 2 wherein the diluent agent is selected from the group consisting of microcrystalline cellulose, lactose monohydrate and dibasic calcium phosphate.

5. The granule formulation according to claim 2 wherein the disintegrant agent is selected from the group consisting of sodium glycolate starch, crospovidone and sodium croscarmellose.

6. The granule formulation according to claim 1 wherein the lubricant agent is selected from the group consisting of glyceryl behenate, glyceryl palmitoestearate and macrogol.

7. The granule formulation according to claim 1 wherein the active ingredient comprises quetiapine hemifumarate.

8. The granule formulation according to claim 1 comprising:
a) a core comprising quetiapine hemifumarate, microcrystalline cellulose, sodium starch glycolate and povidone; and
b) a coating layer comprising glyceryl behenate.

9. The granule formulation according to claim 1 comprising the lubricant agent in an amount of from about 5% to about 25% by weight, based on the total weight of the granule formulation.

10. A process for the preparation of a granule formulation as defined in claim 1 comprising:
a) providing quetiapine or a pharmaceutically acceptable salt thereof, optionally in mixture with a disintegrant agent and/or a diluent agent, as an active ingredient composition;
b) combining the active ingredient composition with binder agent and solvent to form a mixture, comprising a combination sequence selected from among (i) and (ii):
(i) adding the binder agent to the active ingredient composition to form a binder-containing composition, and adding solvent to the binder-containing composition to form said mixture; and
(ii) adding a solution or suspension comprising the binder agent and solvent to the active agent composition to form said mixture;
c) wet granulating said mixture to form granules;
d) drying said granules to form dried granules;
e) sieving said dried granules to recover product granules; and
f) coating the product granules with a lubricant agent, to yield said granule formulation.

11. The process according to claim 10 wherein said solvent comprises a solvent composition selected from the group consisting of water, alcohols and hydroalcoholic mixtures.

12. The process according to claim 10 wherein the wet granulating is conducted in a low shear mixer.

13. The process according to claim 10 wherein said drying is conducted so that said dried granules have a humidity content below 5%.

14. A method of making a pharmaceutical composition, comprising formulating same with a granule formulation according to claim 1.

15. A pharmaceutical composition comprising a granule formulation according to claim 1, optionally in combination with at least one pharmaceutically acceptable excipient.

16. The pharmaceutical composition according to claim 15 in an immediate release form.

17. The pharmaceutical composition according to claim 15 in a sustained release form.

18. The pharmaceutical composition according to claim 15 in a tablet form.

19. An immediate release tablet comprising a granule formulation according to claim 1, wherein the lubricant agent has a concentration in a range of from 5 weight % to 10 weight %, based on total weight of the granule formulation.

20. A sustained release tablet comprising a granule formulation according to claim 1, wherein the lubricant agent has a concentration in a range of from 15 weight % to 25 weight %, based on total weight of the granule formulation.

21. A process for the preparation of a tablet selected from among (i) an immediate release tablet comprising a granule formulation according to claim 1, wherein the lubricant agent has a concentration in a range of from 5 weight % to 10 weight %, based on total weight of the granule formulation, and (ii) a sustained release tablet comprising a granule formulation according to claim 1, wherein the lubricant agent has a concentration in a range of from 15 weight % to 25 weight %, based on total weight of the granule formulation, said process comprising:
(a) preparing a granule formulation according to claim 10, wherein said granule formulation is optionally mixed with at least one pharmaceutically acceptable excipient;
(b) tableting said granule formulation optionally mixed with at least one pharmaceutically acceptable excipient, to form tablets containing said granule formulation; and
(c) coating said tablets.
